(19) United States
(12) Patent Application Publication
(10) Pub. No.: US 2004/0151680 A1
(43) Pub. Date: Aug. 5, 2004

(54) Cosmetic compositions containing phenyl silicones

(76) Inventors: Anjali Abhimanyu Patil, Westfield, NJ (US); Joseph Frank Calello, Bridgewater, NJ (US); Robert Walter Sandewicz, Monroe Township, NJ (US)

Correspondence Address: JULIE BLACKBURN, REVLON CONSUMER PRODUCTS CORPORATION, 237 PARK AVENUE, NEW YORK, NY, 10017, US

(21) Appl. No.: 10/358,868

(22) Filed: Feb. 5, 2003

(51) Int. Cl. A61K 7/06; A61K 7/11

(52) U.S. Cl. 424/70.12

(57) ABSTRACT

Cosmetic compositions containing certain phenyl silicones and the use of such silicones in improving the properties of cosmetic compositions.

### TECHNICAL FIELD

The invention is in the field of cosmetic compositions for application to skin.

### BACKGROUND OF THE INVENTION

Cosmetic formulators are always interested in improving the properties of cosmetic compositions. While a specific cosmetic product may fill one need gap, that product will often create another need gap. The most notable example of this is with the transfer resistant lipsticks. Prior to the invention of these products one need gap in the lipstick field was that lipsticks came off the lips too readily. Cosmetic companies formulated the so-called transfer resistant lipsticks. While these formulas provided excellent transfer resistance and the lipsticks stayed on the lips for extended periods of time, the cosmetic finishes were very matte in texture and in some cases they could be drying to the lips. Accordingly, a completely new need gap was created, namely the desire for lipsticks that were transfer resistant, moisturizing, and at the same time providing a shiny finish.

The use of phenyl substituted silicones is well known in the cosmetic art. For example, U.S. Pat. No. 5,556,613 teaches anhydrous silicone oil based cosmetic compositions containing a homogeneous fatty phase which contains phenyl substituted silicones that have repeating diphenyl siloxy or phenyl trimethylsiloxy moieties in combination with a ethylene wax. The patentee contends that this mixture provides improved anhydrous powder type products.

U.S. Pat. No. 6,136,332 teaches that transfer resistance of cosmetic compositions can be improved by incorporation of certain phenyl substituted silicones into the compositions. These phenyl substituted silicones contain either diphenylsiloxy or phenyltrimethylsiloxy repeating units.

However, it has been discovered that a certain type of phenyl substituted silicone provides improved properties when incorporated into cosmetic compositions. In particular, these specific phenyl substituted silicones provide improved cushion, shine, and compatibility to the formulas. These phenyl silicones may be effective in both transfer resistant and regular, moisturizing color cosmetic compositions.

It is an object of the invention to provide cosmetic compositions with improved payoff, feel, cushion, and internal compatibility but without sacrificing benefits such as transfer resistance.

### SUMMARY OF THE INVENTION

The invention is directed to a cosmetic composition comprising a phenyl substituted silicone selected from the group consisting of A, B, C, D, E, and mixtures thereof, wherein A is:

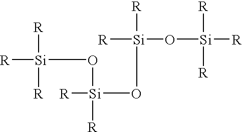

wherein each R is independently methyl or phenyl, with the proviso that the compound contains at least six phenyl groups.

Preferably, A is of the formula:

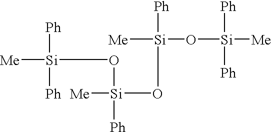

and B is:

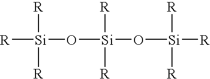

wherein each R is independently methyl or phenyl, with the proviso that the compound contains at least five phenyl groups.

Preferably B is of the formula:

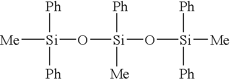

and C is:

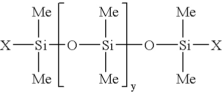

wherein X is —CH₂—CH(CH₃)(Ph); Me is methyl and Ph is phenyl; and y ranges from 1 to 1,000,000,

D is

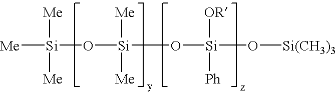

wherein OR′ is SiMe₃, and y is 1-1000; and

E is a phenyl substituted polymer having the general formula:

[R(CH₃)₂—SiO_{1/2}]x[SiO_{4/2}]_{y}

wherein R is phenyl propyl, and x and y are each independently from 1 to about 1,000,000.

The invention is further directed to a method for improving shine, payoff, and feel of a cosmetic composition comprising formulating such composition with an effective amount of a silicone selected from the group consisting A, B, C, D, E, and mixtures thereof.

### DETAILED DESCRIPTION

All percentages are percentages by weight unless otherwise indicated. The phenyl substituted silicones used in the compositions and methods of the invention are selected from the group consisting of A, B, C, D, E, and mixtures thereof wherein A is:

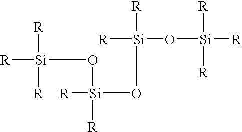

wherein each R is independently methyl or phenyl, with the proviso that the compound contains at least six phenyl groups.

Preferably A is of the formula:

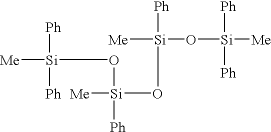

and B is:

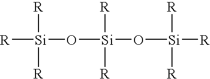

wherein each R is independently methyl or phenyl, with the proviso that the compound contains at least five phenyl groups.

Preferably B is of the formula:

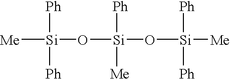

and C is:

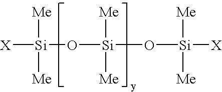

wherein X is —CH₂—CH(CH₃)(Ph); Me is methyl and Ph is phenyl; and y ranges from about 1 to 1,000,000,

and D is:

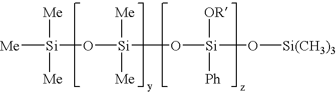

wherein OR′ is SiMe₃ and y is 1-1000; and

E is a polymer having the general formula:

[R(CH₃)₂—SiO_{1/2}]x[SiO_{4/2}]_{y}

wherein R is phenyl propyl, and x and y are each independently from 1 to about 1,000,000.

The silicones may be purchased from a variety of commercial sources. Suitable sources for silicones A and B include Dow Corning, preferably Dow Corning 555 Cosmetic Fluid having the INCI name trimethyl pentaphenyl trisiloxane, which is a mixture of about 60 to 90 parts trimethylpentaphenyltrisiloxane and from about 10 to 30 parts hexaphenyltetrasiloxane. Suitable sources for Silicone C include GE Silicones, preferably SF 1555, which has the INCI name bis-phenylpropyl dimethicone. A suitable source for the D silicone is Wacker-Belsil. A suitable source for the E silicone is General Electric.

One or more of the silicones may be incorporated into a variety of cosmetic compositions, as further described herein. Suitable ranges of the phenyl silicones are from about 0.01-99%, preferably about 0.05-80%, more preferably about 0.1-75% by weight of the total composition.

The phenyl silicones may be incorporated into a variety of anhydrous pigmented cosmetic compositions such as lipsticks, blush, eyeshadow, concealer, nail enamel, and the like. Such compositions may be in the liquid, semi-solid, or solid form. In the preferred embodiment of the invention, the phenyl silicones are incorporated into lipsticks. Preferably such lipsticks are in the semi-solid or solid stick form and contain in addition to the phenyl silicone, pigments, oils, and a structuring agent.

The phenyl silicones may also be suitable for use in a variety of emulsion cosmetic compositions that may be in the liquid, semi-solid, or solid form. The emulsion composition may be a water-in-oil or oil-in-water emulsion comprising about 0.1-95%, preferably about 0.5-85%, more preferably about 1-75% water and about 0.5-55%, preferably about 1-45%, more preferably about 1.5-35% oil. Such emulsion compositions may be in the form of pigmented emulsion compositions such as foundation makeup, cheek color, etc., or skin creams or lotions.

Suitable ingredients that may be found in the pigment anhydrous cosmetic compositions and the emulsion compositions include those set forth below.

A. Pigments and Particulate Fillers

The compositions may comprise from about 0.001-80%, more preferably 0.01-75%, more preferably about 0.5-60% by weight of the total composition of pigments, particulate fillers, or combinations thereof.

1. Organic Pigments

The pigment may comprise organic pigments. Suggested ranges, if present are from about 0.05-70%, preferably about 0.1-25%, more preferably about 0.5-20% by weight of the total composition. The organic pigments may be red, green, blue, yellow, violet, orange, and mixtures thereof. Also suitable are Lakes of such pigments, which means that the organic pigments are reacted with a metal salt such as calcium, aluminum, barium, zirconium, and the like to form salts. Formation of the metal salt of the organic pigment will generally convert the pigment from a water soluble pigment into a water insoluble pigment. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof.

Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors which are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51.

Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

2. Inorganic Pigments

The composition may comprise one or more inorganic pigments. Suitable ranges include about 0.001-55%, preferably about 0.005-10%, more preferably about 0.01-8% by weight of the total composition. Suitable inorganic pigments include iron oxides such as red, blue, black, green, and yellow; titanium dioxide, bismuth oxychloride, and the like. Preferred are iron oxides.

3. Particulate Fillers

It may also be desirable to include one or more particulate fillers in the claimed composition. If so, suggested ranges are about 0.001-40%, preferably about 0.05-35%, more preferably about 0.1-30% by weight of the total composition. Preferably, the particulate filler has a particle size of 0.02 to 100, preferably 0.5 to 100, microns.

Suitable particle fillers include titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature.

B. Oils

A variety of oils may be incorporated into the compositions including organic oils, silicone oils, and mixtures thereof. The oils may be volatile or non-volatile. The term “volatile” means that the oil has a vapor pressure of at least about 2 mm. of mercury at 20° C. The term “non-volatile” means that the oil has a vapor pressure of less than about 2 mm. of mercury at 20° C. The term “oil” means an ingredient that is a pourable liquid at room temperature.

1. Volatile Oils

Suitable volatile liquids generally have a viscosity of about 0.5 to 10 centipoise at 25° C. Suitable volatile oils include linear silicones, cyclic silicones, paraffinic hydrocarbons, or mixtures thereof. If present, suggested ranges of volatile oil are from about 0.1-75%, preferably about 0.5-70%, more preferably about 0.5-65% by weight of the total composition.

(a). Volatile Silicones

Cyclic silicones are of the general formula:

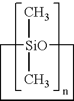

where n=3-6.

Linear volatile silicones in accordance with the invention have the general formula:

(CH₃)₃Si—O—[Si(CH₃)₂—O]ₙ—Si(CH₃)₃

where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

(b). Paraffinic Hydrocarbons

Also suitable as the volatile oil are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

2. Nonvolatile Oils

The composition may contain one or more non-volatile oils such as silicones, esters, and the like. The type of nonvolatile oil may vary depending on the type of cosmetic composition being formulated. If it is desired to formulate more long wearing or transfer resistant lipsticks it is best to use nonvolatile oils having a lower viscosity. On the other hand, if it is desired to formulate lipsticks with more occlusive films it is desirable to use higher viscosity oils. Generally, the lower viscosity nonvolatile oils having a viscosity ranging from about 11-1000, preferably less than 100 centipoise, most preferably less than about 50 centipoise at 25° C. Suitable ranges of non-volatile oil are from about 0.1-95%, preferably about 0.5-90%, more preferably about 1-85% by weight of the total composition.

(a). Silicones

Examples of non-volatile oils include silicones such as polyalkylsiloxanes, and polyarylsiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by reference. Further nonlimiting examples of such silicones include dimethicone, phenyl trimethicone, and the like. Preferred compositions contain a mixture of non-volatile oils which are silicones and organic oils. If present, suggested ranges of non-volatile silicone range from about 0.1-90%, preferably about 0.5-75%, more preferably about 1-65% by weight of the total composition.

(b). Organic Oils

Also suitable are organic oils including saturated or unsaturated, substituted or unsubstituted branched or linear or cyclic organic compounds that are pourable liquids under ambient conditions. Preferred organic oils include those described in U.S. Pat. Nos. 5,505,937; 5,725,845; 5,019,375; and 6,214,329, all of which are incorporated by reference herein in their entirety. Suitable silicone compatible organic esters are mono-, di-, and triesters. The composition may comprise one or more esters selected from the group, or mixtures thereof. Preferably the composition contains at least one of a mono-, di-, or triester. Preferred ranges are about 0.1-85%, preferably about 0.5-80%, more preferably about 1-60% by weight of the total composition of ester.

1. Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the is formula R—OH wherein R is a straight or branched chain saturated or unsaturated alkyl having 2-30 carbon atoms, or phenyl. Both the alcohol and the acid may be substitued with one or more hydroxyl groups, and in one preferred embodiment of the invention the acid is an alpha hydroxy acid. Either one or both of the acid or alcohol may be a “fatty” acid or alcohol, ie. may have from about 6 to 22 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

2. Diesters

Suitable diesters that may be used in the compositions of the invention are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be subsituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer diilinoleate, dicetyl adipate, diisocetyl adipate, diisostearyl dimer dilinoleate, disostearyl fumarate, and so on.

3. Triesters

Suitable triesters comprise the reaction product of a tricarboxylic acid and an aliphatic or aromatic alcohol. As with the mono- and diesters mentioned above, the acid and alcohol may contain from 2 to 30 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, tri C12-13 alkyl citrate, tricaprylin, tricaprylyl citrate, tridecyl behenate, trioctyldodecyl citrate, triisostearyl citrate, tridecyl behenate, tridecyl cocoate, tridecyl isononanoate, and so on. Preferred is a triester which is the reaction product of an citric acid and one or more fatty alcohols, in particular triisostearyl citrate. Also preferred is an ester which is the reaction product of an alpha hydroxy acid and a guerbet alcohol having 6 to 30 carbon atoms, in particular the reaction product of citric acid and octyldodecyl alcohol, referred to as trioctyldodecyl citrate.

4. Hydrocarbon Oils.

Suitable non-volatile hydrocarbon oils used in the compositions include isoparaffins and olefins having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, mineral oil, pentahydrosqualene, squalene, squalane, and mixtures thereof.

5. Lanolin Oil

Also suitable for use in the composition is lanolin oil or derivatives thereof such as hydroxylated lanolin, isobutylated lanolin oil, acetylated lanolin, acetylated lanolin alcohol, and so on.

C. Structuring Agents

The compositions may comprise one or more structuring agents, particularly if it is desired to thicken or solidify the composition. In general, the term “structuring agent” means an ingredient that is a semi-solid or solid at room temperature and which provides viscosity or gelling capability to the formulation. Suitable structuring agents include animal, vegetable, or mineral waxes, silicone waxes, and synthetic polymeric or non-polymeric gelling agents. Suggested ranges of structuring agents, if present, range from about 0.1-60%, preferably 1-40%, more preferably 3-20% by weight of the total composition. Preferably, such structuring agents include waxes, more preferably those that have melting points ranging from about 39 to 135° C., preferably in the range of 45 to 95° C., most preferably 55 to 95° C.

Examples of waxes in accordance with the invention include bayberry, beeswax, candelilla, camauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic camauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like.

Preferably the structuring agent component of the cosmetic composition will contain a mixture of synthetic waxes and natural waxes in a range of about 01.1-50% preferably about 1-20% synthetic wax and about 0.5-20%, preferably 1-15% animal or vegetable wax.

Particularly preferred is where the synthetic wax is an ethylene homopolymer or ethylene copolymer. The molecular weight of the ethylene homopolymer and/or copolymers used as the wax component may vary, so long as the melting point of the homo- or copolymer either alone or in combination is not greater than 135° C. Generally polyethylene waxes having a melting point range of 30 to 135° C. will have a molecular weight ranging from about 100 to about 2,000. Preferably the ethylene copolymers are comprised of ethylene monomer units in either repetitive or randon sequence, in combination with monomer units derived from an ethylenically unsaturated comonomer of the following formula:

CH₂═CH—R₁

wherein R₁ is a C₁₋₃₀ straight or branched chain saturated or unsaturated alkyl, aryl, or aralkyl, preferably a C₁₋₁₀ straight or branched chain alkyl. Examples of ethylene homo- and copolymers which may be used in the invention are set forth in U.S. Pat. No. 5,556,613, which is hereby incorporated by reference.

D. Suspending Agents

It may also be desirable to include one or more suspending agents in the cosmetic composition. Such suspending agents will act to suspending the pigments and particulates present, in addition to working synergistically with any structuring agent present to provide a composition that is homogeneous and resists separation or syneresis.

Suggested ranges of such suspending agents are from about 0.01-60%, preferably about 0.05-50%, more preferably about 0.1-45% by weight of the total composition. Suitable viscosity modifiers include natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

Also suitable as the suspending agent are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl

E. Surfactants

The compositions in accordance with the invention, if in the emulsion form, may exist in two separate phases that are emulsified upon shaking. Preferably the compositions of the invention which are in the emulsion form contain an effective amount of one or more surfactants that cause the dispersed phase to remain emulsified in the continuous phase to form an emulsion having stability which is suitable for commercial purposes. It is also possible that if the compositions of the invention are in the anhydrous form they may contain one or more surfactants to help emulsify the various ingredients found in the anhydrous composition.

Suggested ranges of surfactants are about 0.01-20%, preferably about 0.1-15%, more preferably about 0.5-10%. Suitable surfactants include silicone surfactants or organic surfactants, which may be anionic, cationic, nonionic, zwitterionic, or amphoteric. Preferably the surfactants are nonionic organic or silicone surfactants.

1. Silicone Surfactants

Preferred are nonionic silicone surfactants having at least one hydrophilic radical and at least one lipophilic radical. These silicone surfactants may be a liquid or solid at room temperature and are water-in-oil or oil-in-water type surfactants which have an Hydrophile/Lipophile Balance (HLB) of about 2 to 18. Preferably the silicone surfactant is a nonionic surfactant having an HLB of about 2 to 12, preferably about 2 to 10, most preferably about 4 to 6. The HLB of a nonionic surfactant is the balance between the hydrophilic and lipophilic portions of the surfactant and is calculated according to the following formula:

HLB=7+11.7×log M_{w}/Mₒ

where M_{w} is the molecular weight of the hydrophilic group portion and Mₒ is the molecular weight of the lipophilic group portion.

The polymeric silicone surfactant used in the invention may have any of the following general formulas:

MₓQ_{y}, or

MₓT_{y}, or

MDₓD′_{y}D″_{z}M

wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula R′SiO_{1.5} or RSiO_{1.5} wherein R is methyl and R′is a C₂₋₂₂ alkyl or phenyl. Q is a quadrifunctional siloxy unit having the empirical formula SiO_{4/2} and D, D′, D″, x, y, and z are as set forth below, with the proviso that the compound contains at least one hydrophilic radical and at least one lipophilic radical. Preferred is a linear silicone of the formula:

MDₓD′_{y}D″_{z}M

wherein M=RRRSiO_{1/2}

D and D′=RR′SiO_{2/2}

D″=RRSiO_{2/2}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein

M=trimethylsiloxy

D=Si[(CH₃)][(CH₂)ₙCH₃]O_{2/2} where n=0-40,

D′=Si [(CH₃)][(CH₂)ₒ—O-PE)]O_{2/2} where PE is (—C₂H₄O)ₐ(—C₃H₆O)_{b}H, o=0-40,

a=1-100 and b=1-100, and

D″=Si (CH₃)₂O_{2/2}

More specifically, suitable silicone surfactants have the formula:

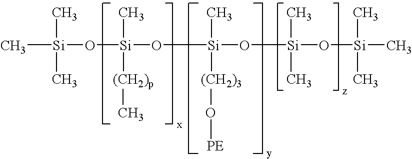

wherein p is 0-40, and

PE is (—C₂H₄O)ₐ(—C₃H₆O)_{b}—H

where x, y, z, a, and b are such that the maximum molecular weight of the polymer is approximately 50,000.

Another type of preferred organosiloxane emulsifier suitable for use in the compositions of the invention are emulsifiers sold by Union Carbide under the Silwet™ trademark, which are referred to by the CTFA term “dimethicone copolyol”.

Also suitable as nonionic silicone surfactants are hydroxy-substituted silicones such as dimethiconol, which is defined as a dimethyl silicone substituted with terminal hydroxy groups.

Examples of suitable silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

2. Organic Surfactants

Also suitable for use are one or more organic surfactants, preferably nonionic organic surfactants. Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, laureth, 1-100 where the number of repeating ethylene oxide units is 1 to 100, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as the nonionic surfactant are alkyoxylated carboxylic acids, which are formed by the reaction of a carboxylic acid with an alkylene oxide or with a polymeric ether. The resulting products have the general formula:

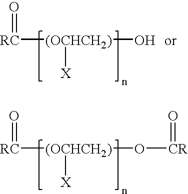

where RCO is the carboxylic ester radical, X is hydrogen or lower alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula:

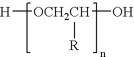

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

In the preferred compositions of the invention, the nonionic surfactant is selected from an nonionic organic surfactant, in particular a nonionic silicone surfactant, more specifically dimethicone copolyol.

F. Skin Conditioning Agents

The compositions comprise one or more skin conditioning agents in ranges from about 0.01-20%, preferably about 0.1-15%, more preferably about 0.5-10% by weight of the total composition. The skin conditioning agents are capable of moisturizing skin without promoting oil secretion or contributing to oil on the skin surface. In particular, it is well known that oily skin can still be dry due to inadequate moisture in the skin tissue. The skin conditioning agents used herein address the moisture and hydration needs of skin and have no negative impact on skin oils. Suitable skin conditioning agents include a variety of organic compounds and polymers.

1. Quaternary Ammonium Compounds

Particularly suitable for use as skin conditioning agents are quaternary ammonium compounds. These ingredients may be monomeric or polymeric and are positively charged tetra-substituted nitrogen derivatives having the following general structure:

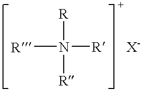

wherein R, R′, R″ and R′″ may be the same or different but may not be hydrogen, and R, R′, R″, and R′″ are selected from C₁₋₃₀ straight or branched alkyl, and wherein X′ represents an anion such as chloride, ammonium, methosulfate, and the like.

Preferred are quatemary ammonium polymers referred to as Polyquatemium having a numerical designation from 1 to 51. Examples of such polymers are Polyquatemium-4 which is a copolymer of hydroxyethylcellulose and diallyldimethyl ammonium chloride, Polyquatemium-5 which is a copolymer of acrylamide and bet-methacrylyloxyethyl trimethyl ammonium methosulfate, Polyquatemium-6 which is a copolymer of dimethyl diallylammonium chloride, Polyquatemium-7 which is a polymeric quaternary ammonium salt consisting of acrylamide and dimethyldiallyl ammonium chloride, Polyquatemium-8 which is a polymeric quaternary ammonium salt of methyl and stearyl dimethylaminoethyl methacrylate quaternized with dimethyl sulfate, and so on. Particularly preferred is Polyquatemium-51 which is a copolymer of butyl-2-methyl-2-propenoate and an ester of orthphosphoric acid. Polyquatemium-51 has the chemical name 3,5,8-triox-4-phosphaundec-10-en-1-aminium, 4-hydroxy-N,N,N, 1-tetramethyl-9-oxo, inner salt 4-oxide polymer with butyl-2-methyl-2-propenoate. Polyquatermium 51 is available from Collaborative Laboratories under the tradename Lipidure.

2. Alcohols

Various aliphatic or aromatic mono-, di- or polyfunctional organic alcohols may be used as skin conditioning agents in the composition of the invention. Generally, such alcohols have the formula R—OH wherein R is a straight or branched C₂₋₃₀ alkyl or C₂₋₃₀ alkyl amido alkyl, or C₂₋₃₀ alkyl amido alkoxy. Preferably R has one or more substituted hydroxyl groups, making R—OH polyhydric. Examples of such alcohols include butylene glycol, ethylene glycol, glycerin, propylene glycol, panthenol, panthenyl ethyl ether, and phytantriol. Panthenol is a trihydric alcohol of the formula R—OH wherein R is an alkyl amido alkyl having two substituted hydroxyl groups. It has the formula:

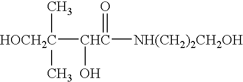

Panthenyl ethyl ether, a monohydric alcohol, is the ethyl ether of panthenol having the formula:

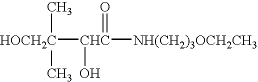

Phytantriol is an aliphatic polyhydric alcohol having the general formula:

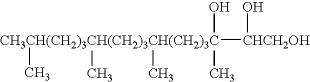

In certain types of compositions it is desirable that they contain one or more skin conditioning agents comprising about 0.01-5% of the quaternary ammonium compound and about 0.01-10% of one or more alcohols.

G. Film Forming Ingredients

The compositions in accordance with the invention may comprise one or more film formers that, upon drying, will aid in forming a continuous cosmetic film on the skin. The film former may be present in an amount of about 0.1-45%, preferably about 0.5-20%, more preferably about 1-15% by weight of the total composition. The film formers may advantageously be resinous plant extracts or synthetic polymers.

1. Resinous Plant Extracts

Examples of resinous plant extracts that provide film forming properties include materials such as rosin and shellac, celluosics including nitrocellulose, celluose acetate propionate, celluose acetate butyrate, or derivatives thereof.

2. Synthetic Polymeric Film Formers

Suitable synthetic polymers may be silicone or organic based. Particularly suitable are siloxy silicate polymers having the following general formula:

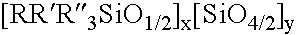

wherein R, R′ and R″ are each independently a C₁₋₁₀ straight or branched chain alkyl, and x and y are such that the ratio of (RR′R″)₃SiO_{1/2} units to SiO₂ units is 0.5 to 1 to 1.5 to 1.

Preferably R, R′ and R″ are each a C₁-6 alkyl, and more preferably are methyl and x and y are such that the ratio of (CH₃)₃SiO_{1/2} units to SiO_{4/2} units is about 0.75 to 1. One type of siloxy silicate is trimethylsiloxy silicate containing 2.4 to 2.9 weight percent hydroxyl groups, which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 2-0749 and 2-0747, which is a blend of about 40-60% volatile silicone and 40-60% trimethylsiloxy silicate. Dow Corning 2-0749 in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

Also suitable are synthetic polymers that are often found in the form of an aqueous dispersion where the polymer particles are dispersed in the aqueous phase of the polymer emulsion. Examples of such polymers include homo- or copolymers of monomers such as acrylic acid, methacrylic acid or C₁₋₃₀ esters of acrylic or methacrylic acid, vinyl pyrrolidone, vinyl acetate, urethane, amides, C₁₋₃₀ hydroxy esters of acrylic or methacrylic acid, vinyl isodecanoate, styrene, and olefins such as ethylene, propylene, butene, pentene, decene, hexadecene, and so on. Such film forming polymers may be used as is or in the form of an aqueous emulsion of solvated or dispersed particles.

H. Finish Enhancers

The composition may contain one or more compounds that enhance the finish of the composition after it is applied to skin. Preferred finish enhancers are synthetic elastomers which may be silicone elastomers or organic polymers having elastomeric properties. The term “elastomer” means a compound exhibits properties associated with rubber such as extensibility with applied force, retractibility upon release of the force, and lack of permanent deformation as a result of extension. Rubber like properties are generally seen in high molecular weight cross-linked polymers having weak intermolecular forces. Preferred elastomers are generally in the solid particulate form having particle size ranging from about 0.05 to 75 microns. The claimed compositions preferably comprise about 0.1-25%, preferably about 0.5-15%, more preferably about I-10% of one or more elastomers. Elastomers provide a velvety smooth finish to the composition, improved spreadability and blendability, and a light, non-greasy feel.

1. Synthetic Organic Polymeric Elastomers

A variety of cross-linked synthetic polymeric elastomers may be used as finish enhancers, including those polymerized from various types of ethylenically unsaturated monomers such as acrylic acid, methacrylic acid, and simple esters thereof, vinyl monomers such as vinyl acetate, vinyl isodecanoate, methyl vinyl ether; maleic anhydride. These monomers may be copolymerized with one or more organic compounds such as esters, glycols, fatty acids, and so on. Examples of such polymers include acrylatesNVA crosspolymer, acrylates/vinyl isodecanoate crosspolymer, adipic acid/diethylene gleyol/glycerin crosspolymer, allyl methacrylates crosspolymer, HDI/trimethylolhexyllactone crosspolymer, lauryl acrylate/VA crosspolymer, methyl methacrylate crosspolymer, PVM/MA decadiene crosspolymer, PEG crosspolymer, PPG-35/PPG-51 glyceryl ether/IPDI crosspolymer, trimethyl pentanediol/adipic acid/glycerin crosspolymer, and so on. Also suitable is HDI/trimethylolhexyllactone crosspolymer which is a crosslinked condensation polymer formed from the reaction of hexyldiisocyanate with the esterification product of trimethylolpropane with 6 to 7 moles of hexyllactone. This polymer is available from Kobo Products under the tradename BPD-500 which is a combination of silicate and the polymer having the INCI name HDI/trimethylol hexyllactone crosspolymer (and) silica. It is a fine white powder having a particle size of about 5-20 microns comprising about 95-99% polymer and 1-5% silica.

2. Silicone Elastomers

Also suitable for use as finish enhancers are silicone elastomers such as those disclosed in U.S. Pat. No. 6,171,581 which is hereby incorporated by reference in its entirety. Examples of such elastomers include cetearyl dimethicone/vinyl dimethicone crosspolymer, dimethicone copolyol crosspolymer, dimethicone crosspolymer, dimethicone/phenyldimethicone crosspolymer, dimethicone/vinyl dimethicone crosspolymer, and mixtures thereof

I. Solvents

The silicones set forth herein may be incorporated into nail enamel compositions. If so, such compositions generally comprise one or more organic solvents. Suggested ranges include about 0.01-95%, preferably 0.5-80% by weight of the total composition. Suitable organic solvents include acetone, C1-4 alkyl acetates, isopropanol, ethanol, glycol ethers, and the like. Glycol ethers are typically ethers or ether esters of alklyene glycols such as ethylene, propylene, dipropylene, glycols and C₁₋₂₀ acids.

J. Other Ingredients

The compositions of the invention may comprise a variety of additional ingredients such as preservatives, antioxidants, botanicals, and the like.

In general, suitable lipstick compositions comprise, by weight of the total composition, about 0.001-70% pigments, about 0.1-75% of one or more oils, and optionally 0.1-30% of one or more structuring agents as well as other optional ingredients as set forth herein.

Suitable nail enamel compositions generally comprise from about 0.01-80% organic solvent, and about 0.1-45% of one or more film forming polymers, and optionally 0.001-70% pigments. Also, such nail enamel compositions may be water based, and if so at least a portion of the organic solvent is replaced with water. Suitable nail enamel compositions are as set forth in U.S. Pat. Nos. 5,066,484; 5,772,988; and U.S. Patent Publication No. 2002/0018759, all of which are hereby incorporated by reference in their entirety. Compositions such as blush, eyeshadow, concealer, tattoos, and the like may be in the anhydrous or aqueous emulsion form. Such compositions generally comprise from about 0.001-70% pigments, optionally from 0.1-75% oils, 0.1-30% structuring agents, and other optional ingredients including water if the compositions are in the aqueous form.

Creams and lotions are most often in the emulsion form and generally comprise water and oil in the ranges set forth above, in addition to optional ingredients such as film forming polymers, botanical extracts, skin conditioning agents, and so on in the ranges set forth herein.

The invention will be further described in connection with the following examples which are set forth for the purposes of illustration only.

### EXAMPLE 1

A transfer resistant lipstick composition was prepared according to the following formula:

| Ingredient | % by weight |
|---|---|
| Polyethylene | 14.00 |
| Cyclomethicone (D5) | 40.00 |
| Trimethylpentaphenyl trisiloxane | 10.00 |
| Trioctyldodecyl citrate | 2.50 |
| Lanolin | 9.70 |
| Quaternium 18 hectorite | 2.00 |
| Methyl paraben | 0.30 |
| Propyl paraben | 0.10 |
| BHT | 0.10 |
| D&C Red #27 (33% in lanolin) | 6.80 |
| Red iron oxide (50% in lanolin) | 13.50 |
| Mica | 1.00 |

The composition was prepared by combining the oils and waxes and heating to a temperature sufficient to melt the waxes. Separately, the pigments were ground in a portion of the oil componet, and added to the heated mixture. The ingredients were mixed well and cooled to room temperature.

### EXAMPLE 2

A traditional moisturizing lipstick composition was made according to the following formula:

| Ingredient | % by weight |
|---|---|
| Polyethylene | 8.50 |
| Microcrystalline wax | 2.00 |
| Trimethylpentapheny trisiloxane | 20.00 |
| Octyldodecyl neopentanoate | 10.00 |
| Lanolin | 9.70 |
| Triisostearyl citrate | 28.00 |
| Methyl paraben | 0.30 |
| Propyl paraben | 0.10 |
| BHT | 0.10 |
| D&C Red #27 (33% in lanolin) | 6.80 |
| Red iron oxide (50% in lanolin) | 13.50 |
| Mica | 1.00 |

The composition was made in the same manner as set forth in Example 1.

### EXAMPLE 3

Foundation makeup compositions were prepared as follows:

| | w/w % | | | |
|---|---|---|---|---|
| | 1 | 2 | 3 | |
| 1 Cyclomethicone¹ | 19.00 | 18.50 | 21.24 | (oil) |
| 1 Bis phenylpropyl dimethicone | 1.00 | 1.00 | 1.00 | (oil) |
| 1 Titanium dioxide/methicone (ultrafine)² | 1.00 | 1.00 | 2.00 | (pigment) |
| 1 Titanium dioxide/methicone² | 8.00 | 8.00 | 6.00 | (pigment) |
| 1 Silica | 1.00 | 1.00 | 1.00 | (pigment) |
| 1 Nylon-12 | 2.00 | 2.00 | 2.00 | (pigment) |
| 1 Yellow iron oxide/methicone | 0.98 | 0.98 | 1.12 | (pigment) |
| 1 Red iron oxide/methicone/boron nitride | 0.62 | 0.62 | 0.98 | (pigment) |
| 1 Black iron oxide/methicone | 0.11 | 0.11 | 0.17 | (pigment) |
| 1 Zinc oxide/dimethicone | 2.05 | 2.05 | 2.05 | (pigment) |
| 1 Boron nitride | 1.69 | 1.69 | 1.51 | (pigment) |
| 1 Talc/methicone | 1.60 | 1.60 | 1.72 | (pigment) |
| 1 HDI/trimethylol hexyllactone crosspolymer³ | 2.50 | 2.50 | 2.00 | (synthetic elastomer) |
| 2 Cyclomethicone/dimethicone copolyol⁴ | 5.00 | 6.00 | 5.50 | (oil/surfactant) |
| 2 Phytantriol | 0.10 | 0.10 | 0.10 | (skin conditioner) |
| 2 Cyclomethicone/dimethicone⁵ | 1.00 | 1.50 | 0.50 | (oil) |
| 2 Cyclomethicone/trimethylsiloxysilicate⁶ | 5.00 | 5.00 | 5.00 | (oil/film former) |
| 3 Panthenyl ethyl ether | 0.30 | 0.30 | 0.50 | (skin conditioner) |
| 3 Water | 36.99 | 36.99 | 35.20 | |
| 3 Panthenol | 0.30 | 0.30 | 0.30 | (skin conditioner) |
| 3 Sodium chloride | 0.50 | 0.50 | 0.50 | (emulsion stabilizer) |
| 3 Trisodium EDTA | 0.01 | 0.01 | — | (preservative) |
| 3 Tetrasodium EDTA | — | — | 0.01 | (preservative) |
| 4 Butylene glycol | 2.00 | 2.00 | 2.00 | (humectant) |
| 4 Methylparaben | 0.25 | 0.25 | — | (preservative) |
| 4 Polyquaternium-51 | — | — | 0.50 | (skin conditioner) |
| 5 Ethyl alcohol | 5.00 | 4.00 | — | (volatile solvent) |
| 6 Butylene glycol/mushroom extract | 2.00 | 2.00 | 2.00 | (skin conditioner/ |
| | | | | mushroom extract) |
| 7 Phenoxyethanol/parabens | — | — | 1.00 | (preservative) |
| 7 Methyldihydrojasmonate | — | — | 0.30 | (fragrance) |

¹Dow Corning 245 fluid, Dow Corning Corporation.
²Color Techniques.
³Kobo Products Inc.
⁴3225C Formulation Aid, Dow Corning Corporation. A mixture of 90 parts D4/D5 cyclomethicone (decamethylcyclopentasiloxane and decamethylcyclotetrasiloxane) and 10 parts of dimethicone copolyol.
⁵Brooks Industries Gel Base Sil.
⁶Dow Corning 749 Fluid, a mixture of 50 parts trimethylsiloxysilicate and 50 parts of a mixture of volatile silicones comprised of D4/D5 cyclomethicone.

In a main beaker the Sequence 1 ingredients are combined and mixed well until the pigments are fully dispersed. The Sequence 2 ingredients are added to Sequence 1 and mixed well. Meanwhile, the Sequence 3 ingredients are combined in a separate container and mixed well, and the Sequence 4 ingredients are combined in another separate container and mixed well. The Sequence 3 and 4 mixtures are combined with mixing. The Sequence 5 and 6 ingredients are then added to the mixture of Sequence 3 and 4 ingredients and mixed well. Then the beaker container the mixture of Sequences 3, 4, 5, and 6 are combined with the first beaker containing Sequences 1 and 2 and mixed well until emulsified. After emulsification, the Sequence 7 ingredients are added to the composition and mixed well. The composition is poured into glass bottles for storage.

### EXAMPLE 4

A nail enamel composition according to the invention was prepared as follows:

| Ingredient | % by wt. |
|---|---|
| Nitrocellulose | 17.7 |
| Butyl acetate | 22.2 |
| Ethyl acetate | 27.0 |
| Isopropanol | 8.0 |
| Glyceryl tribenzoate | 13.1 |
| Acetyl tributyl citrate | 4.0 |
| Glyceryl triacetate | 1.0 |
| Stearalkonium bentonite | 1.0 |
| 2,5dibutylphenyl-3,5-di-t-butyl-4-hydroxy benzoate | 1.0 |
| 10% Trimethyl pentaphenyl trisiloxane in butyl acetate | 5.0 |

The composition was prepared by combining the ingredient and mixing well. The composition was poured into glass bottles.

### EXAMPLE 5

A nail enamel composition was prepared as follows:

| | |
|---|---|
| Nitrocellulose | 17.7 |
| Butyl acetate | 26.2 |
| Ethyl acetate | 27.0 |
| Isopropanol | 8.0 |
| Glyceryl tribenzoate | 13.1 |
| Acetyl tributyl citrate | 4.0 |
| Glyceryl triacetate | 1.0 |
| Stearalkonium bentonite | 1.0 |
| 2,5dibutylphenyl-3,5-di-t-butyl-4-hydroxy benzoate | 1.0 |
| 10% Phenyl trimethicone in butyl acetate | 1.0 |

The ingredients were combined and mixed well. The composition was poured into glass bottles.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition comprising a phenyl silicone selected from the group consisting of A, B, C, D, E, and mixtures thereof, wherein A is: 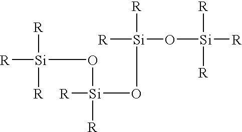
wherein each R is independently methyl or phenyl, with the proviso that the compound contains at least six phenyl groups;
and B is: 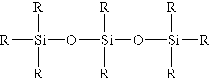
wherein R is methyl or phenyl, with the proviso that the compound contains at least five phenyl groups;
and C is: 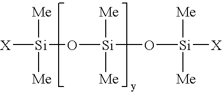
wherein X is —CH₂—CH(CH₃)(Ph); Me is methyl and Ph is phenyl; and y ranges from 1 to 1,000,000;
and D is: 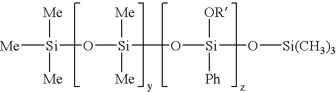
wherein OR′ is SiMe₃, and y is 1-1000; and
E is phenyl substituted polymer having the general formula:
[R(CH₃)₂—SiO_{1/2}]x[SiO_{4/2}]_{y}
wherein R is phenyl propyl, and x and y are each independently from 1 to about 1,000,000;
in a cosmetically acceptable carrier which is an anhydrous pigmented composition or an emulsion composition.

2. The composition of claim 1 wherein the cosmetically acceptable carrier is an anhydrous pigmented cosmetic composition.

3. The composition of claim 2 wherein the composition is in the solid or semi-solid form.

4. The composition of claim 1 wherein the composition is a lipstick, blush, concealer, nail enamel, or eyeshadow.

5. The composition of claim 1 wherein the composition is a solid or semi-solid lipstick.

6. The composition of claim 1 further comprising one or more particulates selected from the group consisting of pigments and powders.

7. The composition of claim 6 wherein the pigments are inorganic pigments, organic pigments, or mixtures thereof.

8. The composition of claim 6 wherein the particulates comprise one or more powders.

9. The composition of claim 1 further comprising one or more oils.

10. The composition of claim 9 wherein the oils are volatile, non-volatile, or mixtures thereof.

11. The composition of claim 10 wherein the volatile oil is a silicone or paraffinic hydrocarbon.

12. The composition of claim 10 wherein the oil comprises a triester.

13. The composition of claim 1 wherein the composition is anhydrous pigmented lipstick composition comprising by weight of the total composition:
0.1-75% volatile oil,
0.1-07% non-volatile oil,
0.1-30% structuring agent, and
0.001-80% of pigments, particulate fillers, or mixtures thereof.

14. The composition of claim 13 wherein the structuring agent is an ethylene polymer.

15. The composition of claim 13 wherein the non-volatile oil is a triester.

16. The composition of claim 15 wherein the triester is trioctyldodecyl citrate.

17. The composition of claim 1 which is an emulsion.

18. The composition of claim 17 which is a pigmented emulsion.

19. The composition of claim 18 which is selected from the group consisting of foundation makeup and nail enamel.

20. A method for improving shine, payoff, and feel of a cosmetic composition comprising formulating such composition with an effective amount of a silicone selected from the group consisting A, B, C, D, E, and mixtures thereof;
wherein A is: 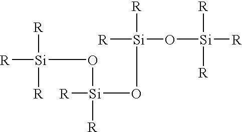
wherein each R is independently methyl or phenyl, with the proviso that the compound contains at least six phenyl groups;
and B is: 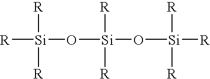
wherein R is methyl or phenyl, with the proviso that the compound contains at least five phenyl groups;
and C is: 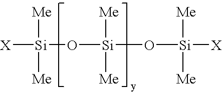
wherein X is —CH₂—CH(CH₃)(Ph); Me is methyl and Ph is phenyl; and y ranges from 1 to 1,000,000;
and D is: 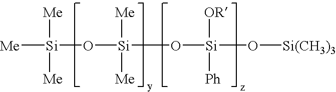
wherein OR′ is SiMe₃, and y is 1-1000; and
E is phenyl substituted polymer having the general formula:
[R(CH₃)₂—SiO_{1/2}]x[SiO_{4/2}]_{y}
wherein R is phenyl propyl, and x and y are each independently from 1 to about 1,000,000;

21. The composition of claim 1 wherein A is:

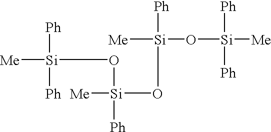

22. The composition of claim 1 wherein B is: [chemical structure]
